(19) United States
(12) Patent Application Publication
(10) Pub. No.: US 2013/0225539 A1
(43) Pub. Date: Aug. 29, 2013

(54) METHOD FOR PRODUCING CRYSTALLINE 5-AMINOSALICYLIC ACID

(71) Applicants: Stefan Gaab, Rosenheim (DE); Alexander Hartung, Wasserburg (DE); Arno Aigner, Tuntenhausen (DE)

(72) Inventors: Stefan Gaab, Rosenheim (DE); Alexander Hartung, Wasserburg (DE); Arno Aigner, Tuntenhausen (DE)

(73) Assignee: Pharmazell GMBH, Raubling (DE)

(21) Appl. No.: 13/822,078

(22) PCT Filed: Sep. 12, 2011
(86) PCT No.: PCT/EP2011/065735

§ 371 Date: May 14, 2013

(30) Foreign Application Priority Data
Sep. 10, 2010 (EP) 10176224.3

(51) Int. Cl. C07C 227/42; C07C 229/64

(52) U.S. Cl. CPC C07C 227/42; C07C 229/64
USPC 514/166; 562/453

(57) ABSTRACT

The invention relates to a method for producing crystalline 5-aminosalicylic acid with a particularly high tap and/or bulk density.

### FIELD OF THE INVENTION

The invention relates to a process for producing crystalline 5-aminosalicylic acid having a particularly high tapped and/or bulk density. Further, the present invention relates to a pharmaceutical composition and certain dosage forms that contain the crystalline 5-aminosalicylic acid according to the invention, and to the use of the crystalline 5-aminosalicylic acid according to the invention for preparing certain dosage forms, and for the therapy and prophylaxis of respective diseases.

### BACKGROUND OF THE INVENTION

Aminosalicylic acids have long been employed as active ingredients in medicaments. Thus, 4-aminosalicylic acid (para-aminosalicylic acid, PAS; IUPAC: 5-amino-2-hydroxybenzoic acid) has been used since the 1940's as an antibiotic in the treatment of tuberculosis, and as a medicament in the treatment of inflammatory bowel diseases (IBD), such as ulcerative colitis and Crohn's disease. Ulcerative colitis is a chronically recurrent inflammation of the large intestine with unknown etiology accompanied by hyperemia, swelling and ulcerations of the mucosa and submucosa. An episodic or continuously progressive course characterized by unforeseen aggravations and remissions is observed. Crohn's disease (regional enteritis) is also a chronic inflammation of unclear origin, but which can affect any part of the intestine, with main localizations in the lower small intestine and/or large intestine. Typically, segments are afflicted in which all layers of the bowel wall are affected, and the formation of fistula and abscesses frequently occurs.

5-Aminosalicylic acid (5-ASA, mesalazine) is employed for the treatment of weakly to moderately active ulcerative colitis. Its pharmacological action is based on topical effects on the intestinal mucosa.

5-ASA is administered in dosages of >3 g per day to achieve the pharmacological protective effect. 5-Aminosalicylic acid is employed in various dosage forms as high dosage formulations in order to have a high bioavailability: these include suppositories, enemas, sachets with micropellets, and tablets. 5-Aminosalicylic acid having a high bulk density is particularly suitable for preparing high dosage formulations in tablet form.

WO-A-01/96280 describes the production of 5-aminosalicylic acid by an electrochemical process at particularly low temperatures, by which advantages for the production could be achieved. Subsequent to an electrochemical step (electrochemical reduction), a raw product is isolated that is converted, after a purification step, to a pure crystalline product in accordance with the specifications of the pharmacopoeias relating to chemical quality. According to the requirements for the respective galenic formulation, the raw product is subsequently converted to the pure product by crystallization under various crystallization conditions.

From the prior art it is known that the temperature and concentration, in particular, in addition to the precipitation time, have a great influence on the formation of the crystal sizes. The application of different crystallization conditions consequently results in different grain size distributions and in materials having different densities. For example, mainly finer crystals are obtained at a low crystallization temperature, whereas coarser crystals are rather obtained at a higher crystallization temperature. At a higher temperature, the large crystals grow while the small crystals dissolve, in accordance with the rules for the behavior of substances in crystallization processes. This is detectable under an optical microscope according to the prior art (cf., FIGS. 1 and 2).

In the crystallization of 5-aminosalicylic acid, acicular crystals basically form in different grain size distributions under different temperature conditions.

For example, as expected fine crystals are obtained at lower temperatures while coarse crystals are obtained at high temperatures. Crystallization can be effected from an aqueous solution through the addition of an aprotic or protic polar, water-miscible solvent at from 0 to 100% by weight, based on the aqueous solution of ASA. Acetone, ethanol, methanol or isopropanol may be employed as the solvent. The temperature range to be applied is from 25° C. to 150° C.

The acicular (needle-shaped) crystals generally have a low bulk and tapped density, because the needles become entangled because of their structure and cannot be packed in an ideal way, as can be shown in FIGS. 3 and 4 using two acicular crystal fractions having different grain size distributions as examples. The acicular structure also causes a poor flowing behavior which is disadvantageous for galenic processing.

### SUMMARY OF THE INVENTION

Thus, it is the object of the present invention to provide a process by which a crystalline 5-ASA can be obtained that meets the requirements of enabling high doses and having a high bioavailability in terms of tapped and/or bulk density when used as an active ingredient in corresponding dosage forms.

In conjunction with the crystallization of 5-aminosalicylic acid it has now surprisingly been found that crystals having unusually high tapped and/or bulk densities can be obtained from a 5-ASA having a coarse crystal structure by a comminuting step that affects the aspect ratio of the crystals. This unforeseen effect is achieved by adjusting the crystallization temperature to the concentration of the substrate and the precipitation times, for example, in water as the solvent, in combination with a technological comminuting step in a state of suspension during or after complete crystallization of a suitable starting quality (cf., FIG. 9). As shown in FIG. 9, the comminuting may be effected from a container into a receptacle, or be performed in a circulation operation in order to save the additional container.

The adjusting of the crystallization temperature mainly favors the longitudinal growth of the crystals. According to the invention, the step of wet grinding adjusts the aspect ratio in such a way that a high bulk density and/or tapped density can be achieved from a particularly coarse particle size distribution. For this purpose, it is required to adjust the required crystallization parameters and thereby obtain a coarse starting material so that the grinding has the desired effect.

Accordingly, the present invention relates to:

1. a process for producing crystalline 5-aminosalicylic acid (5-ASA) having a high bulk and/or tapped density, said process comprising the following steps: (i) crystallizing 5-ASA from an aqueous solution with or without the addition of an aprotic or protic polar, water-miscible solvent in a concentration range of from 0 to 100% w/w at a temperature of from 25° C. to 150° C. and at a pH-value of from 3.0 to 5.0 to form a suspension of 5-ASA; and

(ii) wet grinding the suspension;

2. the process according to item 1, characterized in that said crystallizing is effected at a temperature of from 60° C. to 120° C.;

3. the process according to item 1, characterized in that said crystallizing is effected at a temperature of from 75° C. to 115° C.;

4. the process according to item 1, characterized in that said crystallizing is effected at a temperature of from 90° C. to 110° C.;

5. the process according to any of items 1 to 4, characterized in that the pH-value during crystallization is from 3.5 to 4.5;

6. the process according to any of items 1 to 5, characterized in that said protic solvent is selected from the group consisting of acetone, methanol, ethanol, isopropanol and mixtures thereof;

7. the process according to any of items 1 to 6, characterized in that the suspension has a temperature of <50° C. during said wet grinding;

8. the process according to any of items 1 to 7, characterized in that said wet grinding is effected by means of a Supraton or Ytron mill at flow rates corresponding to a counter-pressure of 1 to 10 bar;

9. the process according to item 1, characterized in that said wet grinding is performed at flow rates corresponding to a counter-pressure of 6 to 9 bar;

10. the process according to any of items 1 to 9, characterized by comprising a further step (iii) of cooling the suspension;

11. the process according to any of items 1 to 10, characterized by comprising a further step (iv) of separating the 5-ASA crystals from the mother liquor;

12. the process according to item 11, characterized in that said separating is effected by means of centrifugation;

13. the process according to any of items 1 to 12, characterized by comprising a further step (v) of drying the 5-ASA crystals;

14. 5-aminosalicylic acid (5-ASA) obtainable by the process according to any of items 1 to 13;

15. 5-aminosalicylic acid (5-ASA) according to item 14, characterized by a bulk density of from 300 g/l to 700 g/l;

16. 5-aminosalicylic acid (5-ASA) according to item 14, characterized by a tapped density of from 510 g/l to 900 g/l;

17. 5-aminosalicylic acid (5-ASA) according to item 14, characterized by a grain size distribution of X(10)=1 μm-30 μm, X(50)=15 μm-60 μm, X(90)=35 μm-220 μm;

18. 5-aminosalicylic acid (5-ASA) according to item 14, characterized by a bulk density of from 300 g/l to 700 g/l, a tapped density of from 510 g/l to 900 g/l, and a grain size distribution of X(10)=1 μm-30 μm, X(50)=15 μm-60 μm, X(90)=35 μm-220 μm;

19. 5-aminosalicylic acid (5-ASA) according to item 18, characterized by a bulk density of from 300 g/l to 400 g/l;

20. 5-aminosalicylic acid (5-ASA) according to item 18, characterized by a tapped density of from 510 g/l to 700 g/l;

21. 5-aminosalicylic acid (5-ASA) according to item 18, characterized by a grain size distribution of X(10)=3 μm-20 μm, X(50)=15 μm-45 μm, X(90)=50 μm-100 μm, preferably by a grain size distribution of X(10)=3 μm-5 μm, X(50)=35 μm-40 μm, X(90)=90 μm-100 μm;

22. 5-aminosalicylic acid (5-ASA) according to item 18, characterized by a bulk density of from 300 g/l to 400 g/l, a tapped density of from 510 g/l to 700 g/l, and a grain size distribution of X(10)=3 μm-20 μm, X(50)=15 μm-45 μm, X(90)=50 μm-100 μm, preferably by a grain size distribution of X(10)=3 μm-5 μm, X(50)=35 μm-40 μm, X(90)=90 μm-100 μm;

23. 5-aminosalicylic acid (5-ASA) according to item 18, characterized by a bulk density of from 400 g/l to 500 g/l;

24. 5-aminosalicylic acid (5-ASA) according to item 18, characterized by a tapped density of from 600 g/l to 800 g/l;

25. 5-aminosalicylic acid (5-ASA) according to item 18, characterized by a bulk density of from 400 g/l to 500 g/l, preferably above 400 g/l to 500 g/l, a tapped density of from 600 g/l to 800 g/l, and a grain size distribution of X(10)=5 μm-25 μm, X(50)=25 μm-50 μm, X(90)=50 μm-200 μm, preferably by a grain size distribution of X(10)=7 μm-10 μm, X(50)=25 μm-35 μm, X(90)=80 μm-90 μm; 26. suppositories, enemas, sachets with micropellets and tablets comprising the 5-ASA according to any of items 14 to 21;

27. use of the 5-ASA according to any of items 14 to 26 for preparing a dosage form selected from the group consisting of suppositories, enemas, sachets with micropellets and tablets;

28. a pharmaceutical composition comprising the 5-ASA according to any of items 14 to 26;

29. the 5-aminosalicylic acid as defined in one or more of items 14 to 26 for use in the therapy and prophylaxis of a disease selected from the group consisting of Crohn's disease, ulcerative colitis and tuberculosis.

### BRIEF DESCRIPTION OF THE FIGURES

FIG. 1 shows an optical micrograph of crystals of 5-aminosalicylic acid obtained at temperatures of <40° C.

FIG. 2 shows an optical micrograph of crystals of the same compound obtained at temperatures of >40° C.

FIGS. 3 and 4 show examples of particle size distributions of two differently sized acicular crystal fractions obtained according to the prior art by different temperature controls of the crystallization with different grain size distributions according to the following Table 1:

TABLE 1
Examples of Crystalline 5-Aminosalicylic Acids with
Different Physical Characteristics

| | 5-ASA fine acicular | 5-ASA coarse acicular |
|---|---|---|
| Product | crystals | crystals |
| Bulk density | 150-190 g/l | 250-270 g/l |
| Tapped density | 300-390 g/l | 450-480 g/l |
| Particle size distribution | X (10) = 2-6 μm | X (10) = 10-15 μm |
| | X (50) = 8-20 μm | X (50) = 50-60 μm |
| | X (90) = 25-50 μm | X (90) = 200-220 μm |

FIG. 5 shows the particle size distribution of 5-ASA having a high bulk density.

FIG. 6 shows the particle size distribution of 5-ASA having a particularly high bulk density.

FIG. 7 shows an optical micrograph of 5-ASA having a high bulk density.

FIG. 8 shows an optical micrograph of 5-ASA having a particularly high bulk density.

FIG. 9 shows a schematic representation of the wet grinding process, wherein 1=crystallization container, 2=wet grinding, 3=receptacle, and 4=centrifuge/dryer.

FIG. 10 shows the laser diffractogram of 5-ASA having a particularly high bulk density.

FIG. 11 shows the laser diffractogram of 5-ASA having a high bulk density.

FIG. 12 shows the circularity of 5-ASA having a particularly high density.

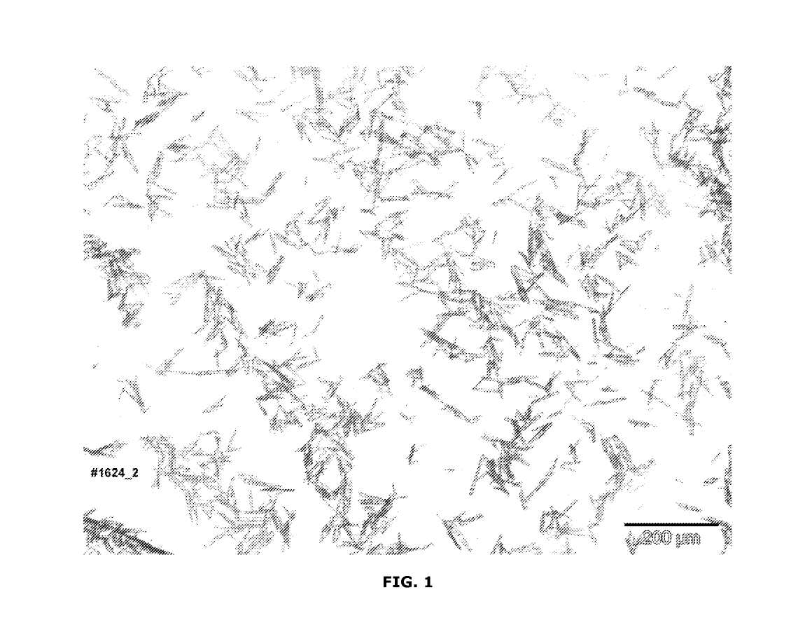

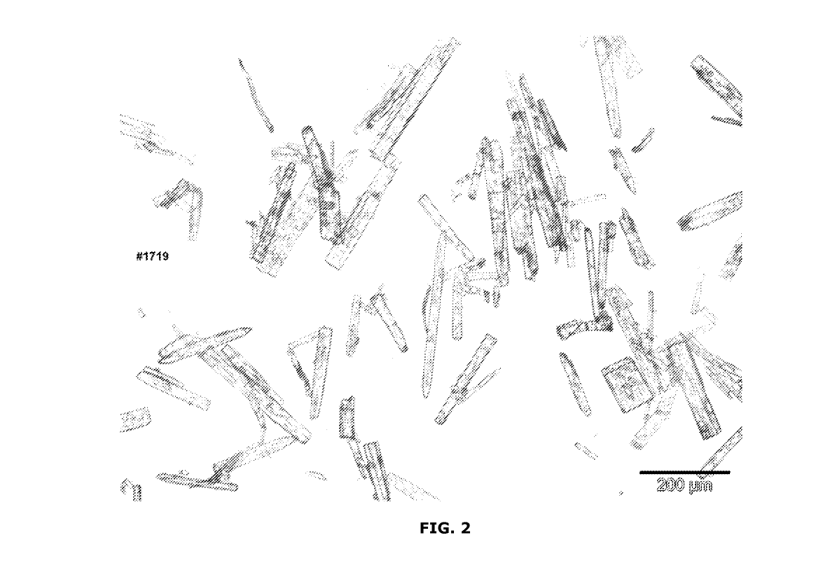

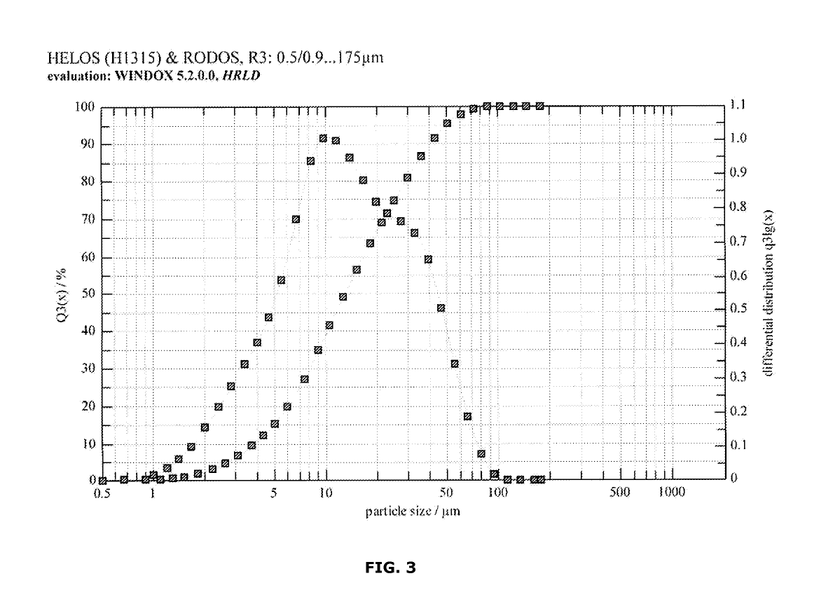

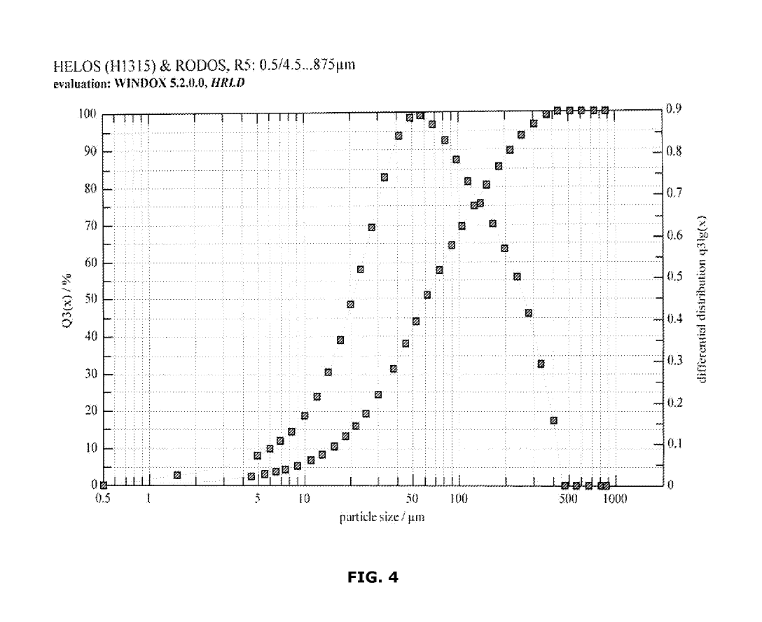

### SUBJECT MATTER OF THE INVENTION

Therefore, the present invention relates to a process for producing crystalline 5-aminosalicylic acid (5-ASA) having a particularly high bulk and/or tapped density, comprising the following steps:

(i) crystallizing 5-ASA from an aqueous solution with or without the addition of an aprotic or protic polar, water-miscible solvent in a concentration range of from 0 to 100% w/w at a temperature of from 25° C. to 150° C. and at a pH-value of from 3.0 to 5.0 to form a suspension of 5-ASA; and (ii) wet grinding the suspension.

In one embodiment according to the invention, the step of crystallizing the 5-ASA is performed at a temperature of from 60° C. to 120° C., and in another embodiment, it is performed at a temperature of from 75° C. to 115° C., and in yet another embodiment, it is performed at a temperature of from 90° C. to 110° C.

In another preferred embodiment, the pH-value during crystallization is from 3 to 4.5.

The protic polar solvent is selected from the group consisting of various water-miscible alcohols, such as methanol, ethanol, propanol, isopropanol, butanol, isobutanol, sec-butanol and tert-butanol, or mixtures thereof. Methanol, ethanol and isopropanol are preferred.

The aprotic polar solvent is selected from the group consisting of water-miscible ketones, such as acetone, 2-butanone, 2-pentanone and 3-pentanone, or mixtures thereof. Acetone is preferred.

Mixtures of at least one protic polar and at least one aprotic polar solvent may also be employed. Mixtures of acetone, methanol or/and ethanol are preferred.

The adjusting of the crystallization temperature mainly favors the longitudinal growth of the crystals, as could be shown above. Then, the step of wet grinding adjusts the aspect ratio of the crystals in such a way that a high bulk density can be achieved from a coarse starting material which does not yet show the desired properties. For this purpose, it is required to adjust the required concentration range to from 2% to 12% w/w and the temperature range to from 25° C. to 150° C. and thereby obtain a coarse starting material so that meanwhile or subsequently the wet grinding has the desired effect. For higher concentrations of the solution to be crystallized, the temperature must be increased to achieve the required proportion of coarse particles.

A suitable coarse starting material is crystalline 5-ASA as prepared under the conditions of the intermediate steps stated in Examples 1 and 2, the crystalline properties of which being similar to those of the coarse crystalline 5-ASA stated in Table 1.

After the wet grinding step, the suspension containing 5-ASA crystals has a temperature of <150° C. in one embodiment and, in another embodiment, from 25 to 100° C.

The devices for grinding used for performing the process according to the invention can be constituted of a rotor-stator system, wherein the rotor which is also referred to as the tool, can have different shapes and designs.

The special characteristic of the wet grinding step according to the invention is that this process employs no mills in a classical sense for this purpose, but apparatus or devices that are commercially available as homogenizers and through which the desired effect according to the invention can be surprisingly achieved by the superposition of different physical effects whose overall influence cannot be calculated in advance or otherwise predicted.

Homogenization technology is based on the application of high-pressure relaxation to liquids which further comminutes the predispersed particles. This results in stable dispersions for various applications. The product passes through the high-pressure pump, is condensed and subsequently relaxed again in the homogenizing valve. The associated mechanical energy input causes the desired product properties.

Without being limited by theory, it can be assumed that the physical effects can be considered as superpositions of multistage shear effects in hydrodynamic shearing fields, high frequency oscillating forces, intense mixing of the liquid and solid phase, and pressure built-up.

In the wet grinding step the grinding parameters can be adjusted to achieve the desired effect by adjusting the pressure to from 0 to 20 bar, for example, from 4 to 15 bar. Preferably, Supraton or Ytron homogenizers can be used for this purpose, but other commercially available homogenizers and in-line mills are also suitable for the purposes of the invention.

Suitable homogenizers include, for example, a homogenizer of the Ytron Z series, manufactured by the company Ytron Process Technology GmbH & Co. KG (Bad Endorf, Germany, www.ytron.de), or a homogenizer of the Supraton S series, manufactured by the BWS Technologie GmbH (Grevenbroich, Germany, www.supraton.com). These homogenizers (reactors) include up to five, preferably up to three, rotor/stator sets with an extremely low radial distance. One or more liquid phases and the materials suspended therein are forced to pass through the multirow (5 or 3 rows) sprocket labyrinth.

The wet grinding is preferably effected by means of a Supraton or Ytron homogenizer at flow rates corresponding to a counter-pressure of 1 to 20 bar, preferably from 4 bar to 15 bar.

Preferred further steps following steps (i) and (ii) include (iii) cooling of the suspension obtained by the comminuting step to room temperature (22° C.), optionally (iv) separating the crystals from the mother liquor, preferably by centrifugation, and optionally (v) drying the product, for example, in a spiral dryer, preferably under vacuum at <1000 mbar and at 80° C. The process according to the invention enables crystalline 5-ASA to be obtained with high bulk and/or tapped densities.

Thus, the present invention further relates to a crystalline 5-aminosalicylic acid having a bulk density of from 300 g/l to 700 g/l and/or a tapped density of from 510 g/l to 900 g/l, especially one obtainable by the process according to the invention as described herein.

The 5-ASA according to the invention has a bulk density of from 300 g/l to 700 g/l, in one embodiment from 310 g/l to 600 g/l, and in another embodiment from 330 g/l to 500 g/l.

The tapped density of the 5-ASA according to the invention is within a range of from 510 g/l to 900 g/l, in one embodiment within a range of from 550 g/l to 800 g/l, and in another embodiment within a range of from 600 g/l to 700 g/l.

The grain size distribution of the 5-ASA according to the invention is within ranges of X(10)=1 μm-30 μm, X(50)=15 μm-60 μm, X(90)=35 μm-220 μm, in one embodiment within ranges of X(10)=3 μm-20 μm, X(50)=15 μm-45 μm, X(90)=50 μm-100 μm, in another embodiment within ranges of X(10)=5 μm-25 μm, X(50)=25 μm-50 μm, X(90)=50 μm-200 μm, in another embodiment within ranges of X(10)=3 μm-5 μm, X(50)=35 μm-40 μm, X(90)=90 μm-100 μm, and in another embodiment within ranges of X(10)=7 μm-10 μm, X(50)=25 μm-35 μm, X(90)=80 μm-90 μm.

The bulk density and tapped density are measured by methods known to the skilled person, such as those described in the USP (“United States Pharmacopeia”) monograph, e.g., USP 27, Vol. 1, pp. 226 to 227, May 1, 2009-Apr. 30, 2010, Bulk Density 616, Method 1, Tapped Density 616, Method 2. The measurement of the grain size distribution is effected according to the protocols of European Pharmacopeia, Supplement 6.6, Chapter 2.9.31 “Particle Size Analysis by Laser Light Diffraction” pp. 5103-5107.

Thus, another embodiment of the present invention is a crystalline 5-amino-salicylic acid characterized by a bulk density of from 300 g/l to 700 g/l, a tapped density of from 510 g/l to 900 g/l, and a grain size distribution of X(10)=1 μm-30 μm, X(50)=15 μm-60 μm, X(90)=35 μm-220 μm.

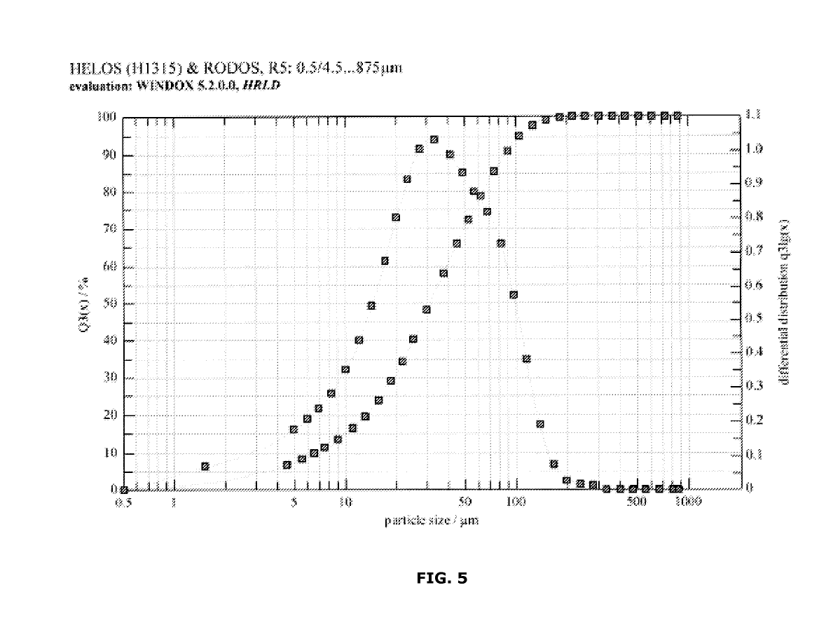

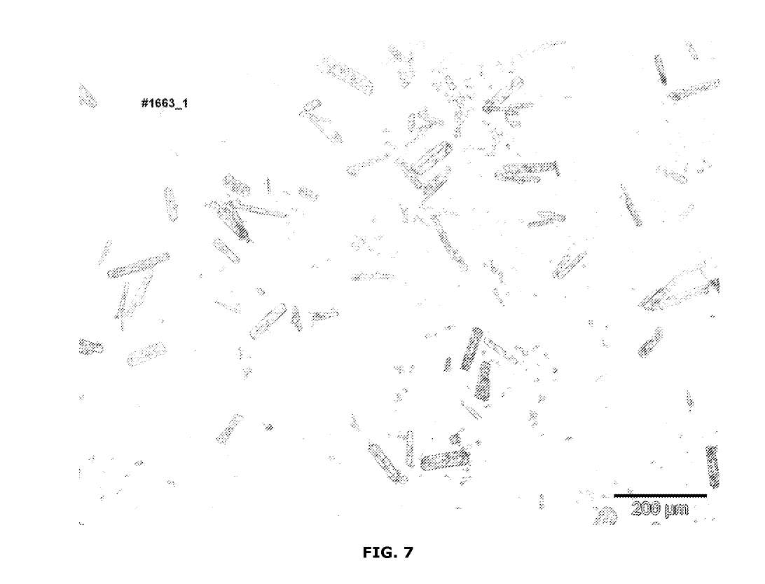

Still another embodiment is a crystalline 5-aminosalicylic acid characterized by a bulk density of from 300 g/l to 400 g/l, a tapped density of from 510 g/l to 700 g/l, and a grain size distribution of X(10)=3 μm-5 μm, X(50)=35 μm-40 μm, X(90)=90 μm-100 μm, as shown by way of example in FIGS. 5 and 7.

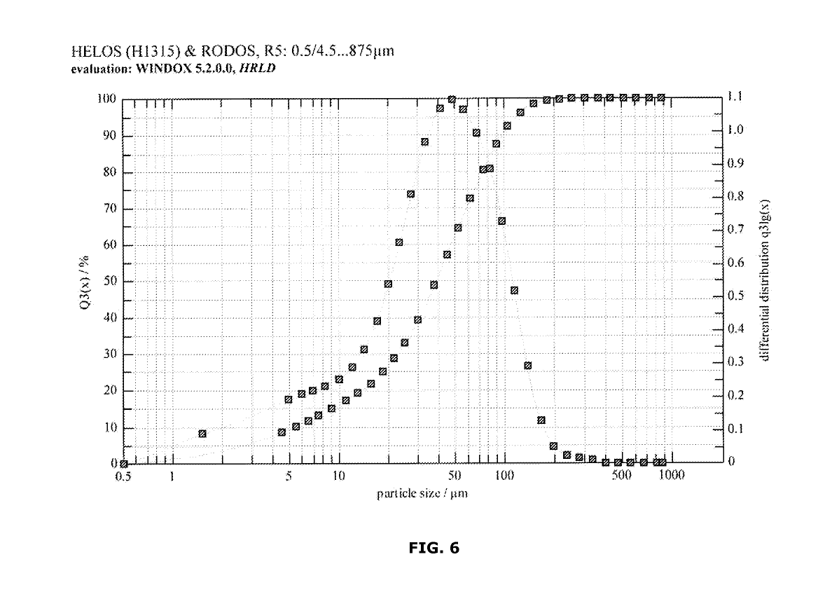

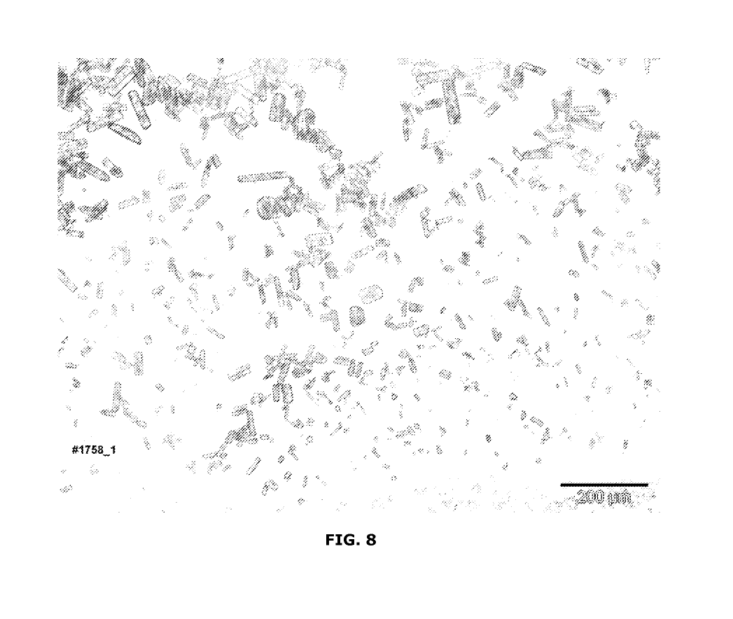

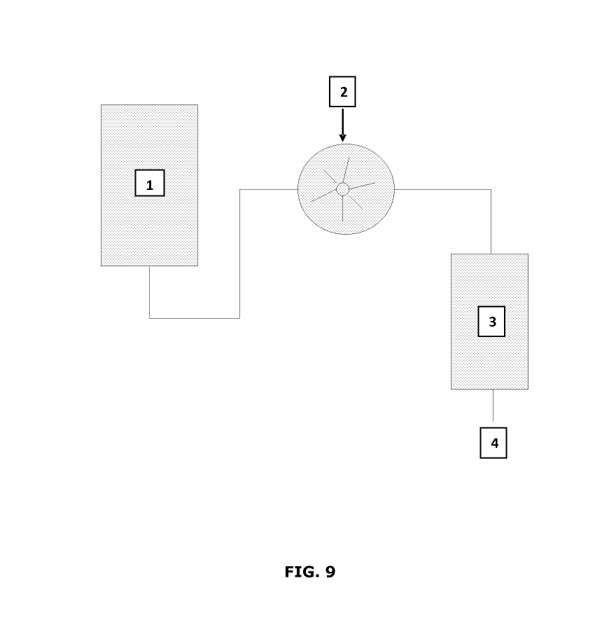

Yet another preferred embodiment is a crystalline 5-aminosalicylic acid characterized by a bulk density of from 400 g/l to 500 g/l, a tapped density of from 600 g/l to 800 g/l, and a grain size distribution of X(10)=7 μm-10 μm, X(50)=25 μm-35 μm, X(90)=80 μm-90 μm, as shown by way of example in FIGS. 6 and 8.

The crystals are described by their particle size distribution as observed by laser diffraction, their optical micrograph image and their aspect ratio which can be determined, for example, by means of image analysis using the Malvern Symex FPIA 3000.

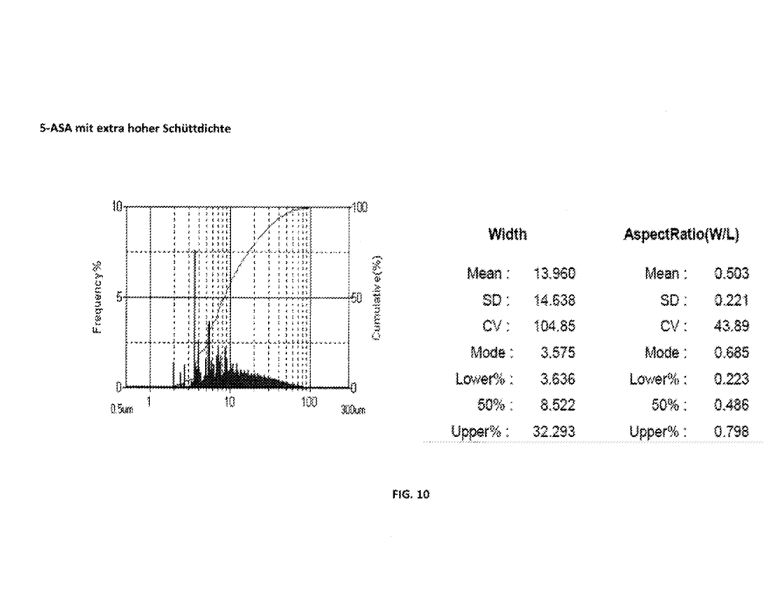

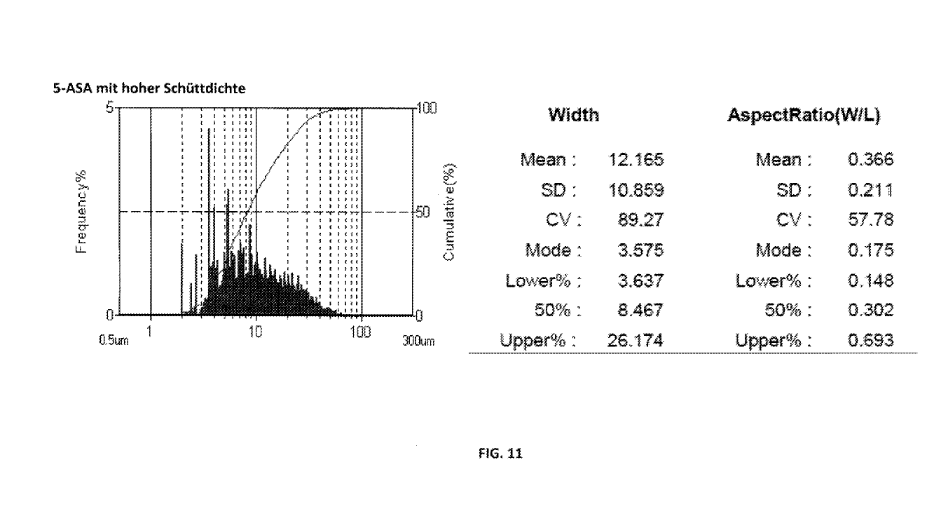

The aspect ratio (width/length; w/l) is expressed by a proportion. In the case of the 5-ASA according to the invention having a particularly high bulk density, the main fraction of the crystals has an aspect ratio around 0.5. From this, a width to length ratio of w/l of 1:2 can be calculated. In the 5-ASA having a high bulk density according to the invention, the aspect ratio is around 0.36, thus corresponding to a width to length ratio of 1:3. The different aspect ratios of the 5-ASA crystal fractions according to the invention having high and particularly high bulk densities bring about the different bulk and tapped densities and the differently broad grain size distributions seen in the laser diffractograms according to FIG. 10 and FIG. 11.

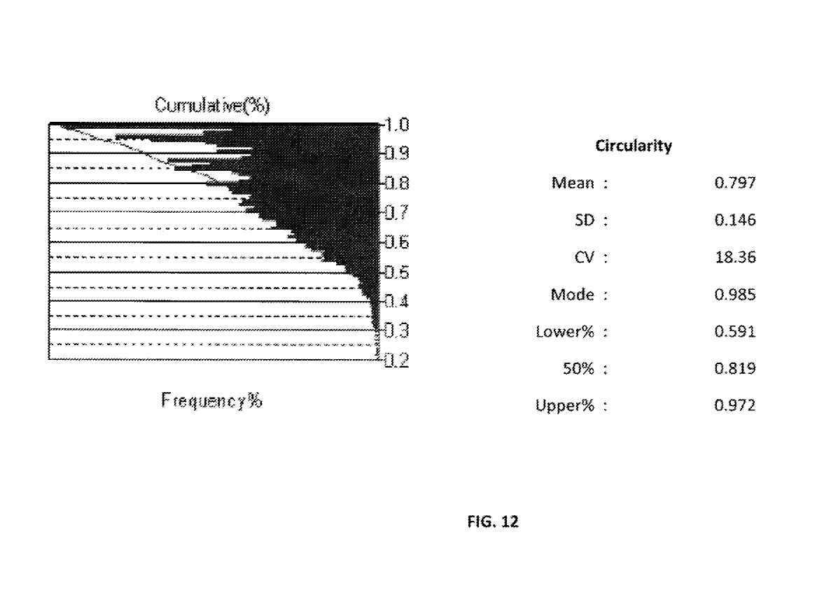

The measured value of circularity describes the deviation of the crystal shape from ideal spherical shape. A value of 1 corresponds to an ideal sphere. The value of almost 0.8 for the circularity of the 5-ASA according to the invention having a particularly high bulk density shows that the shape of the crystals is substantially closer to spherical shape than to acicular shape. This is in turn demonstrated by the high bulk and tapped densities, the comparatively narrow grain size distribution and the resulting favorable galenic processability, like the circularity of the 5-ASA according to the invention having an extremely high density as measured with a Malvern Sysmex FPIA 3000 according to FIG. 12.

In one embodiment, the 5-ASA crystals according to the invention have an aspect ratio of from 1:1.8 to 1:2.2, in another embodiment of 1:2.

In another embodiment according to the invention, the 5-ASA crystals according to the invention have a circularity of from 0.7 to 0.85.

The present invention further relates to pharmaceutical dosage forms, such as suppositories, enemas, sachets with micropellets and tablets, comprising the crystalline 5-ASA according to the invention.

In addition, the present invention further relates to the use of the crystalline 5-ASA according to the invention for preparing suppositories, enemas, sachets with micropellets and tablets.

Also, the present invention further relates to a pharmaceutical composition comprising the crystalline 5-ASA according to the invention.

The present invention further relates to the crystalline 5-aminosalicylic acid according to the invention for use in the therapy and prophylaxis of a disease selected from the group consisting of Crohn's disease, ulcerative colitis and tuberculosis.

The present invention is illustrated by the following Examples without being limited thereto.

### EXAMPLES

### Example 1

About 600 kg of raw mesalazine (5-aminosalicylic acid) is suspended in 2000 l of drinking water, and the suspension which is at about 30° C., is adjusted to pH-value<1 using aqueous hydrochloric acid. Thereupon, the mesalazine is dissolved as a hydrochloride. The solution is treated with 17 kg of active charcoal as a precaution. After the active charcoal was filtered off, the clear solution is slowly adjusted to a pH-value of 3.5 to 4 by the addition of alkali at 90° C. to 110° C. This causes 5-aminosalicylic acid to crystallize in coarse crystals which are then ground at <50° C., either continuously or in a circular flow, by means of a suitable at 10 bar in a state of suspension. A three-stage Supraton of the S 300.7.4 series (manufacturer BWS Technologie, see above) having a three-bladed impeller in the first rotor stage and a gap of 0.3 mm between rotors and stators serves as a comminuting tool. The Supraton is operated with a flow rate of 13 to 19 metric tons per hour and a power consumption of 72 to 85 Ah.

Characteristics of the rotor and stator relationships in a Supraton:

| Rotor 1/Stator 1 | Rotor 2/Stator 2 | Rotor 3/Stator 3 |
|---|---|---|
| three-bladed impeller/ | gap width 7 mm/ | gap width 4 mm/ |
| bolt circle 3 mm | bolt circle 2 mm | bolt circle 1 mm |

Alternatively, an Ytron homogenizer (manufacturer Ytron Process Technology, see above) of the series Z45.00 with a three-stage rotor-stator tool and a gap width of 0.4 mm and slot width of rotor and stator tools of 1.5 mm with the same flow rate as that of the above described Supraton device with 45 Ah power consumption may be used. In the Ytron homogenizer employed, the slot width of the 3rd stator is 1 mm instead of 1.5 mm for stators 1 and 2.

After the grinding, the pH of the suspension is checked again and, if necessary, readjusted, the suspension is cooled down to room temperature and separated from the mother liquor by a centrifuge, and washed thereafter. The product is dried in a spiral dryer under a vacuum [500-1000 mbar/40-80° C]. The yield is about 450 kg of pure product.

### Example 2

About 600 kg of raw mesalazine (5-aminosalicylic acid) is suspended in 2000 l of drinking water, and the suspension obtained which is at about 30° C., is adjusted to pH-value<1 using aqueous hydrochloric acid. Thereupon, the mesalazine is dissolved as a hydrochloride. The solution is treated with 17 kg of active charcoal as a precaution. After the active charcoal was filtered off, the clear solution is heated at 70° C. to 110° C., preferably 80° C. to 90° C., and the pH is slowly adjusted to 3.5 to 4 by the addition of alkali. This causes 5-aminosalicylic acid to crystallize in slightly smaller crystals as compared to Example 1 when the stirrer is rotated at a slow rate, which crystals are then ground at <50° C., either continuously or in a circular flow, by means of a comminuting tool as described above a suitable mill under 10 bar in a state of suspension. The Supraton or Ytron homogenizers as described in Example 1 are used as comminuting tools.

After the grinding, the pH-value is checked again and, if necessary, readjusted, the suspension is cooled down to room temperature and separated from the mother liquor by a centrifuge, and washed thereafter. The product is dried in a spiral dryer under a vacuum [500-1000 mbar/40-80° C]. The yield is about 450 kg of pure product.

The properties of the crystalline 5-aminosalicylic acids obtained in Examples 1 and 2 are compared with those of corresponding commercially available products (fine crystalline 5-ASA and coarse crystalline 5-ASA) in Table 2.

TABLE 2
Comparison of Different Crystalline 5-Aminosalicylic Acids

| | 5-ASA fine | 5-ASA coarse | | |
|---|---|---|---|---|
| Product | crystals*⁾ | crystals*⁾ | Example 1 | Example 2 |
| Bulk density | 150-190 | 250-270 | 400-500 | 300-400 |
| [g/l] | | | | |
| Tapped | 300-390 | 450-480 | 600-800 | 510-700 |
| density [g/l] | | | | |
| Grain size | X(10) = 2-6 | X(10) = 10-15 | X(10) = 7-10 | X(10) = 3-5 |
| distribution | X(50) = | X(50) = | X(50) = | X(50) = |
| [μm] | 8-20 | 50-60 | 25-35 | 15-45 |
| | X(90) = | X(90) = | X(90) = | X(90) = |
| | 25-50 | 200-220 | 80-90 | 90-100 |

*⁾5-ASA fine crystals and 5-ASA coarse crystals are comparative products according to the prior art

As can be seen from Table 2, the crystalline 5-aminosalicylic acids according to the invention prepared by the process according to the invention have higher tapped and bulk densities as compared to the previously available crystalline 5-aminosalicylic acids “5-ASA fine crystals” and “5-ASA coarse crystals” according to the prior art. The crystalline 5-aminosalicylic acids according to the invention are thus particularly suitable for the preparation of medicaments, especially in tablet form, having a high concentration of active ingredient.

### INDUSTRIAL APPLICABILITY

The invention enables the production of 5-aminosalicylic acid having a particularly high tapped density and/or bulk density which allows for a high dosage and bioavailability as an active ingredient in respective dosage forms, especially in the form of tablets.

1. 5-Aminosalicylic acid (5-ASA), characterized by having a bulk density of from 300 g/l to 700 g/l and grain size distribution of X(10)=1 μm-30 μm, X(50)=15 μm-60 μm, X(90)=35 μm-220 μm.

2. 5-Aminosalicylic acid (5-ASA) according to claim 1, characterized by a tapped density of from 510 g/l to 900 g/l.

3. A process for producing the crystalline 5-aminosalicylic acid (5-ASA) as defined in claim 1 comprising the following steps:
(i) crystallizing 5-ASA from an aqueous solution of 5-ASA with or without the addition of protic or aprotic polar solvents in a concentration range of from 0 to 100% at a temperature of from 25° C. to 150° C. and at a pH-value of from 3.0 to 5.0 to form a suspension of 5-ASA; and
(ii) wet grinding the suspension in a homogenizer.

4. The process according to claim 3, characterized in that said crystallizing is effected at a temperature of from 60° C. to 120° C.

5. The process according to claim 3, characterized by comprising a further step (iii) of cooling the suspension.

6. The process according to claim 3, characterized by comprising a further step (iv) of separating the 5-ASA crystals from the mother liquor.

7. The process according to claim 3, characterized by comprising a further step (v) of drying the 5-ASA crystals.

8. Dosage articles selected from the group consisting of suppositories, enemas, sachets with micropellets and tablets comprising the 5-aminosalicylic acid (5-ASA) according to claim 1.

9. Use of the 5-aminosalicylic acid (5-ASA) according to claim 1 for preparing a dosage form selected from the group consisting of suppositories, enemas, sachets with micropellets and tablets.

10. A pharmaceutical composition comprising the 5-aminosalicylic acid (5-ASA) according to claim 1.

11. The 5-aminosalicylic acid as defined in claim 1 for use in therapy and prophylaxis of a disease selected from the group consisting of Crohn's disease, ulcerative colitis and tuberculosis.
